(19) United States
(12) Patent Application Publication
(10) Pub. No.: US 2021/0208045 A9
(43) Pub. Date: Jul. 8, 2021

(54) Method Of Measuring Viscosity In A Microfluidic System

(71) Applicant: Biomillenia SAS, Romainville (FR)

(72) Inventor: Aleksander Dajkovic, Paris (FR)

(21) Appl. No.: 16/621,328

(22) PCT Filed: Jun. 11, 2018
(86) PCT No.: PCT/EP2018/065407

§ 371 Date: Dec. 11, 2019

(30) Foreign Application Priority Data
Jun. 12, 2017 (EP) 17175474.0

(51) Int. Cl. G01N 11/02; C12Q 1/02; G05D 7/06

(52) U.S. Cl. CPC G01N 11/02; G01N 2011/008; G05D 7/0694; C12Q 1/02

(57) ABSTRACT

The invention relates to a microfluidic method for measuring viscosity in a micro droplet in a microfluidic system, comprising the steps of i) introducing a fluorescent molecule into a micro droplet otherwise comprising a fluid, ii) in the microfluidic system, exciting the fluorescent molecule in said micro droplet by applying light to the micro droplet, iii) measuring the resulting fluorescence emitted from the micro droplet thereby determining the viscosity of the fluid in the micro droplet. The invention also relates to method of screening for microorganisms or cells that produce viscosity-modulating compounds with desired properties. Finally, the invention also relates to the use of fluorescent molecules for measuring the viscosity of a fluid in a micro droplet in a microfluidic system.

### FIELD OF THE INVENTION

The present invention is in the field of microfluidic systems and methods that use such systems. More specifically, the microfluidic system is used to measure differences of viscosity in micro droplets contained therein.

### BACKGROUND

Viscosity measurement is traditionally done with viscometers or rheometers whose underlying technologies can be varied (for viscometers: falling sphere viscometer, U-tube viscometers, falling piston viscometers, vibrational viscometers, etc.; for rheometers: dynamic shear rheometers, rotational cylinder, cone and plate, etc.). Typically, fluid volumes between 1 ml and 5 ml are used, and the actual measurement process requires between 1 minute and 5 minutes. All mechanical methods have in common that the fluid is subjected to shear forces, and the resistance of the fluid to these forces (internal friction) is measured. The internal friction of a fluid is proportional to the dynamic viscosity and the velocity gradient (i.e., the shear rate) between layers of different velocities. In all cases, the relatively high amount of sample fluid and the slow measurement process preclude real-time viscosity measurements in small samples or localized regions. Finally, mechanical viscometers are affected by proteins present in biological samples adhering to the surfaces of the instrument. This not only requires scrupulous cleaning between measurements, but may also introduce another source of error through protein deposition during the measurement process.

Besides these drawbacks, the traditional measurement methods are tedious and time-consuming, they require expensive instrumentation, and are restricted to bulk sample sizes.

There is therefore a need in the art for a method that allows to measure the viscosity of compounds without the drawbacks of the known methods.

The inventors have astonishingly found that it is possible to measure the viscosity of tiny droplets in a microfluidic system. They have been able to do this by measuring the fluorescent emission of a fluorophore that is contained inside the droplets. The invention therefore allows to miniaturise the volumes required for viscosity measurements and to massively parallelise the measurement of the viscosity of many different substances.

A related problem is that in the food and in the cosmetics industries viscosity-modulating compounds and microorganisms are often sought as principal components or ingredients in products. Nevertheless, at present, there is no technology that allows to efficiently find such compounds or microorganisms that produce them.

The solution provided by the present invention is to use microfluidic screening of microorganisms or more generally cells that modify viscosity of their surrounding medium. The invention is based on the surprising finding that the droplets can be distinguished from each other based on their viscosity. The inventors have indeed surprisingly found that droplets that comprise fluorophores have a different fluorescence depending on their viscosity. This difference is measurable and allows to select the droplets with the desired viscosity. The invention therefore allows screening large numbers of microorganisms or cells to find those that produce viscosity-modulating compounds with the desired properties.

### BRIEF DESCRIPTION OF THE INVENTION

The invention relates to a microfluidic method for measuring viscosity in a micro droplet in a microfluidic system, comprising the steps of i) providing a micro droplet, wherein the micro droplet comprises a fluid and a fluorescent molecule, ii) in the microfluidic system, exciting the fluorescent molecule in said micro droplet by applying light to the micro droplet, and iii) measuring the resulting fluorescence emitted from the micro droplet thereby determining the viscosity of the fluid in the micro droplet.

The invention also relates to a method of screening for microorganisms or cells that produce viscosity-modulating compounds comprises the following steps: a) providing a composition comprising at least one microorganism or cell, b) optionally subjecting said microorganism or cell to a reaction that leads to a change in the genetic material of at least one microorganism or cell, c) encapsulating the microorganism or cell obtained in step b) into a micro droplet, wherein each micro droplet statistically comprises only one microorganism or cell, d) measuring the viscosity in each of the micro droplets by the method of the present invention (i.e. by measuring the fluorescence of the fluorophore in the micro droplets), and e) optionally isolating the micro droplets with the desired viscosity, thereby isolating microorganisms or cells that produce viscosity-modulating compounds with the desired properties.

The invention further relates to the use of a fluorescent molecule for measuring the viscosity of a fluid in a micro droplet in a microfluidic system.

Herein, a microfluidic system is a “microfluidic device” or “microfluidic chip” or “synthesis chip” or “lab-on-a-chip” or “chip” is a unit or device that permits the manipulation and transfer of microliters or nanoliters or picoliters of liquid into a substrate comprising micro-channels. The device is configured to allow the manipulation of liquids, including reagents and solvents, to be transferred or conveyed within the micro channels and reaction chamber using mechanical or non-mechanical pumps.

Herein, a “flow channel” or “channel” means a microfluidic channel through which a fluid or solution may flow. As is known in the art, such channels may have a cross section of less than about 1 mm, less than about 0.5 mm, less than about 0.3 mm, or less than about 0.1 mm. The flow channels of the present application may also have a cross section dimension in the range of about 0.05 microns to about 1,000 microns, or 0.5 microns to about 500 microns, or about 10 microns to about 300 microns. The particular shape and size of the flow channels will depend on the particular application required for the reaction process, including the desired throughput, and may be configured and sized according to the desired application.

A microfluidic “valve” (or “micro-valve”) as used herein means a device that may be controlled or actuated to control or regulate fluid or solution flow among various components of the microfluidic device, including flow between flow channels, solvent or reagent reservoirs, reaction chamber, columns, manifold, temperature controlling elements and devices, and the like. Such valves are known in the art and include, for example, mechanical (or micromechanical valves), (pressure activated) elastomeric valves, pneumatic valves, solid-state valves, etc.

### DETAILED DESCRIPTION OF THE INVENTION

The invention relates to a microfluidic method for measuring viscosity in a micro droplet in a microfluidic system, comprising the steps of i) providing a micro droplet, wherein the micro droplet comprises a fluid and a fluorescent molecule, ii) in the microfluidic system, exciting the fluorescent molecule in said micro droplet by applying light to the micro droplet, and iii) measuring the resulting fluorescence emitted from the micro droplet thereby determining the viscosity of the fluid in the micro droplet.

The microfluidic system may comprise flow channels and valves.

Detection is conducted in a flow channel by interrogation of passing droplets by a laser and measuring the emitted fluorescence. The emitted fluorescence is then used to calculate the viscosity, or at least to detect a change in viscosity, of the fluid of the micro droplet.

Preferably, the fluorescence emitted by the fluorescent molecule depends on the viscosity of the fluid in the micro droplet.

There are two possible methods for determining the viscosity by measuring a fluorescence signal, and two corresponding classes of fluorescent molecules that can be employed.

First, incorporation of most fluorescent molecules in the droplets coupled with detection of fluorescence anisotropy signal on the microfluidic platform permits the detection of differential viscosities in the droplets. The fluorophores in the droplets undergo rotational diffusion which is inversely proportional to the viscosity of the medium (EinsteinSmoluchowski relation). In medium with increased viscosity rotational diffusion is decreased compared to the medium with lower viscosity. When polarized light is shined on rotating fluorescent molecules a fraction of the emitted signal remains polarized and another fraction of the signal becomes depolarized due to a number of processes, among which the rotational diffusion of fluorophores is a major one. The ratio of polarized to depolarized emitted light is termed fluorescence anisotropy. The anisotropy signal is therefore indicative of the viscosity of the medium.

As a second approach, fluorescent molecules containing molecular rotors can be incorporated in droplets. Molecular rotors are a group of fluorescent molecules that form twisted intramolecular charge transfer (TICT) states upon photoexcitation and therefore exhibit two competing deexcitation pathways: fluorescence emission and non-radiative deexcitation from the TICT state. Since TICT formation is viscosity-dependent, the emission intensity of molecular rotors depends on the solvent's viscosity.

Preferably therefore, i) the fluorescent molecule undergoes rotational diffusion that is inversely proportional to the viscosity of the fluid in the micro droplet, and/or ii) the fluorescent molecule is a molecular rotor that forms twisted intra molecular charge transfer upon photo excitation and therefore exhibits two competing de-excitation pathways, the relative intensities of which differ depending on the viscosity.

Accordingly, measuring the fluorescence emitted from the micro droplet is performed i) by determining the fluorescence anisotropy signal when the fluorescent molecule undergoes rotational diffusion, and/or ii) by measuring the emission intensity when the fluorescent molecule is a molecular rotor.

Preferably the fluorescent molecule is selected from the group of benzonitrile-based fluorophores (such as DMABN (dimethylamino benonitrile), benzylidene malononitriles (such as DCVJ (9-(2,2-Dicyanovinyl)julidine)), stilbenes (such as p-DASPMI), arimethene dyes (such as crystal violet), Viscous Blue 1™, Viscous Blue 2™, Viscous Blue 420™, Viscous Green 1™, Viscous Green 2™, Viscous UV™, Viscous Aqua™, Viscous Red™, Viscous VpH™.

The fluorescent molecule can be introduced into the micro droplet either i) during the formation of the micro droplet, or ii) after formation of the micro droplet by a method such as active of passive droplet fusion with a droplet that contains the dye, or iii) after formation of the micro droplet by a method such as nanoinjection or picoinjection. Nanoinjection or picoinjection can be helped by applying a high voltage (for example 20,000 V and 20,000 Hz). Alternatively, other technologies such as acoustic wave technology can also be used to add reagents to droplets.

Preferably the micro droplet comprises at least one microorganism or one cell. This microorganism or cell is subject to the screening method. Optionally, after a genetic change is introduced into the microorganism or cell, it may produce a desired viscosity-modulating substance which may be detected by a change in viscosity in the fluid surrounding the organism.

The term “microorganism” herein encompasses naked DNA or RNA, viruses, phages, bacteria, yeast, and other kinds of microorganisms.

Suitable microorganisms or cells, which might be transformed/transduced/transfected or mutated to produce a compound of interest include, but are not limited to bacterial strains, archaeal strains, fungal strains, yeast strains, algae, plant protoplasts, prokaryotic or eukaryotic cells, spores, insect cells, insect strains, mammalian cells, including human cells, insect cells, Chinese hamster ovary (CHO) cells, and any other type of cell that can be cultured in a micro droplet. In a preferred embodiment of the invention the microorganism which produces a compound of interest is a bacterial strain, a fungal strain or yeast strain. In a most preferred embodiment, the microorganism is a bacterial or fungal strain.

Ideally, the microorganism is a bacteria and preferably the bacteria is not genetically modified (non-GMO).

Hence, ideally the microorganism or cell influences the viscosity of the fluid in the micro droplet. Ideally, the microorganism or cell influences the viscosity by secreting a substance into the fluid.

Preferably, the micro droplet comprises two immiscible phases. When the micro droplet comprises a microorganism or cell, the microorganism or cell is in the aqueous phase. This phase is then examined for a change in viscosity indicating for example the production of the desired substance.

Preferably, the micro droplet has a volume of between 10 pL to 5000 nL. More preferably, the micro droplet has a volume of between 10 pL and 500 nL, even more preferably between 10 pL and 100 nL, yet more preferably between 10 pL and 50 nL and most preferably between 10 pL and 20 nL.

Droplet-based microfluidics technology has allowed major advances in the screening of microorganisms by significantly increasing throughput and enlarging the range of systems that can be selected. Highly monodisperse droplets of picolitre or nanoliter volume can be made, fused, injected, split, incubated and sorted triggered on fluorescence, often at kHz frequencies. Typically, single bacterial or yeast cells are compartmentalized in droplets of 10 pl 20 nL volume, allowing screening of enzymes expressed intracellularly, on the surface of cells or secreted from cells, with a 1,000-fold increase in speed and a 1-million-fold reduction in volume (and hence cost) compared to robotic microtiter plate-based systems.

Microfluidic devices are powerful tools that allow to miniaturise and to perform a large number of assays in parallel. As a consequence, microfluidic devices are ideal tools to vastly increase the throughput of many types of laboratory assays, such as screenings analyses or in vitro evolution.

Microfluidic devices are essentially networks of small channels used for the precise manipulation of small amounts of fluids. Miniaturised reaction vessels, which are in facts droplets of fluid, flow through the channels of the microfluidic system. Along their flow path, the droplets can be manipulated. A reagent can for example be added to at least a subset of the droplets by various methods known in the art, such as nanoinjection or picoinjection. Such a reagent can for example be a substrate for an enzymatic reaction which becomes fluorescent if an enzyme with desired properties is present in the droplet. In such a setup, the droplets are incubated and the fluorescence of the individual droplets is measured. The droplets with the desired level of fluorescence, can then be selected. This allows for example screening a large number of different enzymes with random mutations.

To date, no system has been developed for measuring the viscosity in such a system in particular the viscosity of fluids in micro droplets in such a system. This is of importance as when screening for certain phenotypes of organisms, some methods require measuring a change in viscosity.

The method of the invention allows for screening for microorganisms or cells that produce viscosity-modulating compounds. The inventors have indeed astonishingly found an efficient way of selecting organisms that produce compounds that have a desirable viscosity.

This will greatly speed up the development of, e.g. but not limited to non-GMO bacteria that produce compounds with desired viscosity.

The present invention therefore also relates to a method of screening for microorganisms or cells that produce viscosity-modulating compounds by means of measuring the viscosity of the fluid around the microorganism in a micro droplet. This screening method is based on the realization by the inventors that the viscosity inside a micro droplet can be inferred from measuring the emission from a fluorophore inside the droplet that changes its emission in response to the viscosity of the medium in which it finds itself. The screening method is therefore based on the method for measuring viscosity in a micro droplet in a microfluidic system described above.

The method of the present invention of screening for microorganisms or cells that produce viscosity-modulating compounds comprises the following steps:
a) providing a composition comprising at least one microorganism or cell,
b) optionally subjecting said microorganism or cell to a reaction that leads to a change in the genetic material of at least one microorganism or cell,
c) encapsulating the microorganism or cell obtained in step a) or b) into a micro droplet, wherein each micro droplet statistically comprises only one microorganism or cell,
d) measuring the viscosity in each of the micro droplets by the method of the present invention (i.e. by measuring a fluorescence of the fluorophore in the micro droplets), and
e) optionally isolating the micro droplets with the desired viscosity, thereby isolating microorganisms or cells that produce viscosity-modulating compounds with the desired properties.

Preferably, subjecting microorganisms or cells to a reaction that leads to a change in the genetic material of at least one of the microorganisms is performed by a reaction involving recombinant DNA technology or CRISPR technology, most preferably by a reaction selected from the group of natural transformation, transduction by phage, conjugation and random mutagenesis.

In molecular biology, transformation is the genetic alteration of a cell resulting from the direct uptake and incorporation of exogenous genetic material from its surroundings through the cell membrane(s). For transformation to take place, the recipient bacteria must be in a state of competence, which might occur in nature as a time-limited response to environmental conditions such as starvation and cell density, and may also be induced in a laboratory.

Transformation is one of three processes for “horizontal gene transfer”, in which exogenous genetic material passes from bacterium to another, the other two being conjugation (transfer of genetic material between two bacterial cells in direct contact) and transduction (injection of foreign DNA by a bacteriophage virus into the host bacterium). In transformation, the genetic material passes through the intervening medium, and uptake is completely dependent on the recipient bacterium.

As of 2014 about 80 species of bacteria were known to be capable of transformation, about evenly divided between Gram-positive and Gram-negative bacteria; the number might be an overestimate since several of the reports are supported by single papers.

“Transformation” may also be used to describe the insertion of new genetic material into nonbacterial cells, including animal and plant cells; however, because “transformation” has a special meaning in relation to animal cells, indicating progression to a cancerous state, the process is usually called “transfection”.

Herein, the invention relates to “natural” transformation.

Natural transformation is a bacterial adaptation for DNA transfer that depends on the expression of numerous bacterial genes whose products appear to be responsible for this process. In general, transformation is a complex, energy-requiring developmental process. In order for a bacterium to bind, take up and recombine exogenous DNA into its chromosome, it must become competent, that is, enter a special physiological state. The DNA integrated into the host chromosome is usually (but with rare exceptions) derived from another bacterium of the same species, and is thus homologous to the resident chromosome.

The capacity for natural transformation appears to occur in a number of prokaryotes, and thus far 67 prokaryotic species (in seven different phyla) are known to undergo this process.

The invention also relates to microorganisms or cells created by such means and identified by measuring the viscosity in the fluid around the organisms.

Among the industrially important lactic acid bacteria only Leuconostoc canosum and Streptococcus thermophillus are demonstrably naturally competent to take up DNA and be transformed (Blonnqvist, Steinmoen, & Havarstein, 2006; Helmark, Hansen, Jellen, Sorensen and Jensen, 2004). The competence of S. themorphillus depends on the growth conditions and the growth medium and a competence stimulating peptide has been discovered (Gardan et al, 2009) and patented (US 2012/0040365 A1).

Essentially, there is a first and a second microorganism. The first carries a trait that one would like to have in another organism, such as the second organism. Sometimes the first organism is merely naked DNA or a phage or a virus. This is encompassed by the invention and the claims.

Preferably, the first of the two or more organisms has the desired trait ab initio and the method serves to detect the transfer of said trait by means of transduction, conjugation or transformation to the second organism lacking the trait.

The method relies on the transfer of the trait encoded in the DNA by means of transformation, transduction or conjugation is actively induced. The trait may be encoded on a plasmid or on the chromosome of the first organism. The first and the second organism are incubated under conditions that allow the transfer of the DNA or RNA from one organism (the first) to the other (the second).

Ideally, the microorganisms are subjected to transformation.

“Random mutagenesis” in the context of this invention comprises any method that allows to introduce mutations into the genetic material of a microorganism or a cell. Many such methods are known in the art and the skilled person will be able to determine which is the most adapted method for each microorganism or cell. Examples of random mutagenesis methods comprise exposure to UV light and treatment by mutagenic chemicals. Random mutagenesis can also be performed during PCR amplification by for example performing error prone PCR or by using nucleotide analogs in the PCR reaction that lead to mis-incorporation of nucleotides. Amplicons generated with such PCR methods can then for example be introduced into microorganisms or cells by transformation or transfection. The amplicons may have to be cloned into a plasmid before this can be performed.

It is preferable not to encapsulate more than one microorganism or cell in any given droplets, as this would make it difficult to ascertain whether and which of the microorganisms and/or cell produces a compound of interest. As a result, each of the micro droplets comprises at most one microorganism or cell. This is generally achieved by producing many more micro droplets than there are cells to be screened. Most of the droplets will in this case be empty and some of them will comprise at most one microorganism or cell.

The screening method according to the invention may in addition comprise a step of incubation after the encapsulation. This may be important to provide the microorganism or cell time to grow or to recover from the mutagenic treatment from optional step b) and to produce a sufficient amount of the viscosity-modulating compound in order for a change in viscosity to be detectable. In one embodiment, the microdroplets in this step are incubated for at least 1 minute, preferably at least 30 minutes, more preferably at least 1 hour, even more preferably at least 2 hours and most preferably at least 24 hours. The ideal incubation time depends on the exact experimental setup and organism.

In one embodiment of the screening method, the incubation temperature is tightly controlled. The ideal incubation temperature mostly depends on the identity of the microorganism or cell. Certain types of bacteria such as E. coli and most mammalian cells for example grow best at a temperature of 37° C. In contrast, yeast cells tend to grow best at 30° C.

In one embodiment of the invention, the micro droplets with the desired viscosity are isolated by fluorescence activated sorting. However other alternatives also exist. The micro droplets can for example be isolated directly on the microfluidic chip by detecting the micro droplets with the desired viscosity by a detector and by isolating the micro droplets of interest for example by applying an electromagnetic field or an acoustic wave to the droplets of interest to push them into an alternative channel of the chip to be collected.

The present invention also relates to the use of a fluorescent molecule for measuring the viscosity of a fluid in a micro droplet in a microfluidic system.

### FIGURE CAPTIONS

FIG. 1

Fluorescent signal from DCVJ (y-axis) from droplets with buffer (low signal on x-axis) and from droplets containing 5 mg/mL of hyaluronic acid.

FIG. 2

Droplet-making chip design showing inlet for fluorinated oil, for aqueous solution containing cells (and possibly a fluorescent molecule), and the outlet for droplets.

FIG. 3

Drawing of a typical design for a droplet nanoinjection device (2).

FIG. 4

Picture of the process of nanoinjection.

### EXAMPLES

### Example 1: Measuring the Viscosity Inside a Droplet by Measuring the Fluorescence

We have undertaken proof of concept experiments for the molecular rotor DCVJ (9-(2,2-Dicyanovinyl)julolidine). We have incorporated this fluorophore in droplets containing buffer and in droplets containing 5 mg/mL hyaluronic acid (solution of 5 mg/mL of hyaluronic acid is highly viscous). To be able to distinguish different droplets, the droplets with buffer were additionally marked with low concentration of sulforhodamine, while the droplets with 5 mg/mL of hyaluronic acid were marked with high concentration of sulforhodamine.

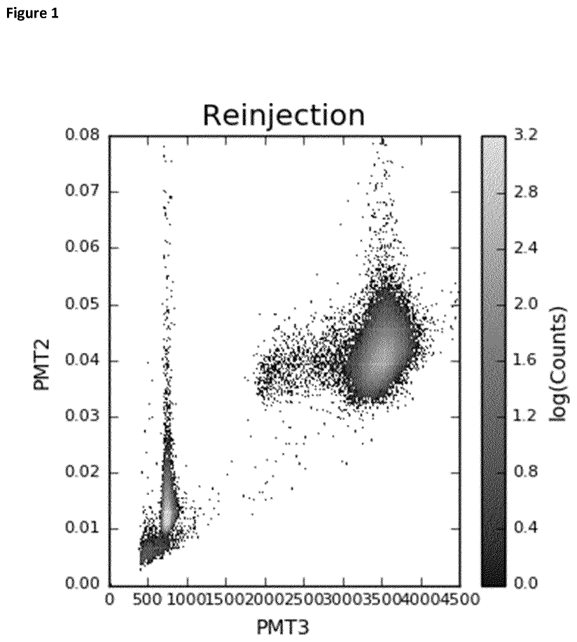

The results are shown in FIG. 1.

The results show that the DCVJ fluorescence is clearly different in droplets containing 5 mg/mL of hyaluronic acid compared to the droplets containing buffer. This shows that the viscosity of the fluid inside the droplet can be calculated by measuring the fluorescence emission of an appropriate fluorophore.

### Example 2: Preparation of a Droplet Generating Device (1)

Examples 2 to 5 provide one experimental setup that can be useful to perform microfluidic viscosity and screening experiments according to the invention.

Soft-lithography in poly(dimenthylsiloxane) (PDMS) was used to prepare the droplet generating device (1). A SU-8 photoresist mould was used to prepare the PDMS. To prepare the SU-8 mold, a layer of SU-8 was spin coated on a silicon wafer. The wafer was covered by a designed mask and exposed to UV for a certain period of time. After full development and baking the wafer, the SU-8 mould was ready for PDMS. The SU-8 thickness for droplet making chip in this example was 200 μm. The droplet volume generated by the chip depends on the SU-8 thickness. To generate nanoliter droplets, the thickness can vary from 80 μm to 500 μm.

The thickness of the SU-8 mould for different types of PDMS chip varies. The SU-8 thickness for droplet nanoinjection chip, droplet sorting chip can for example respectively be 180 μm and 350 μm.

The droplet volume generated by the chip depends on the SU-8 thickness. To generate nanoliter droplets, the thickness can vary from 80 μm to 500 μm.

After preparation of the SU-8 mould, PDMS was cast on the mould and bound to a glass side. The inside part of microfluidic channel was treated by a commercial surface coating agent (Trichloro-(1H,1H,2H,2H-perfluorooctyl)-silane, Sigma-Aldrich) to make the channel surface hydrophobic.

### Example 3: Generation of Fungi Spore-Containing Droplets

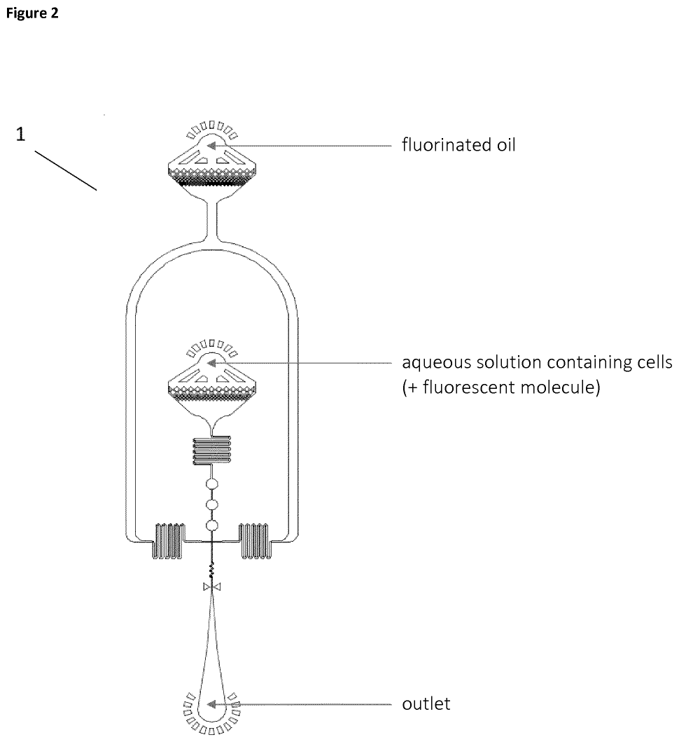

To generate droplets on a chip, the PDMS chip was connected via tubing to an oil phase reservoir, an aqueous phase reservoir and an outlet tubing. A possible chip design is shown in FIG. 2. In this case, the oil phase consists of perfluorocarbon oil (HFE7500, 3M) with 5% (w/w) of a surfactant, made by coupling oligomeric perfluorinated polyethers (PFPE) with polyethyleneglycol (PEG) (Biocompatible surfactants for water-in-fluorocarbon emulsions, Lab Chip, 2008, 8, 1632-1639). However, any phase that is immiscible with the droplets, which in this case are made of an aqueous phase, could have been used (any oil or gas phase). The aqueous phase consists of fungi spore suspension as an example, but is not limited to fungi spores. In other examples, mammalian cells, bacterial cells, yeast cells etc. could be used. The flow rate was controlled by syringe pumps (PHD2000, Havard Apparatus). The flow rate of oil phase was 4 mL/h, and the flow rate of aqueous phase was 3 mL/h. The droplet volume generated here was 20 nL (diameter=0.336 mm). The droplets encapsulate the fungi spores during the droplet generation process. The droplets were collected in a vial and incubated at 30° C. over 48 hours for germination and growth of the fungi.

### Example 4: Nanoinjection of a Fluorescent Molecule into the Droplets

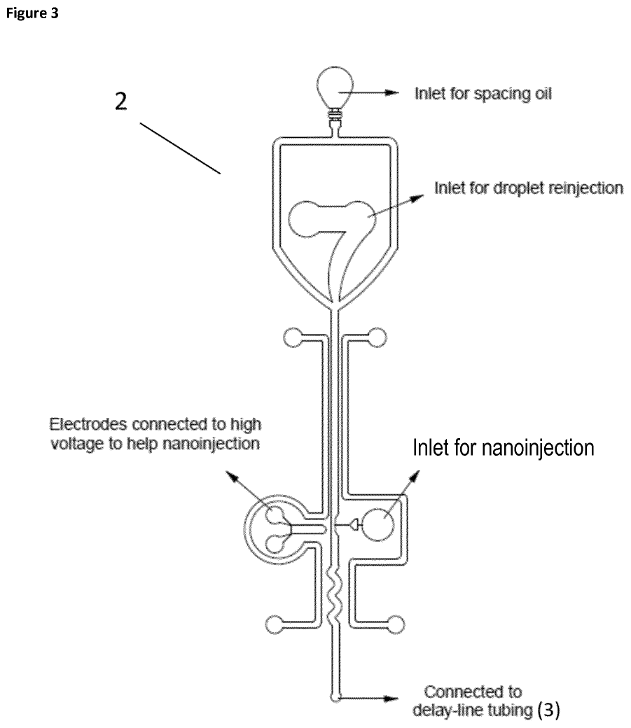

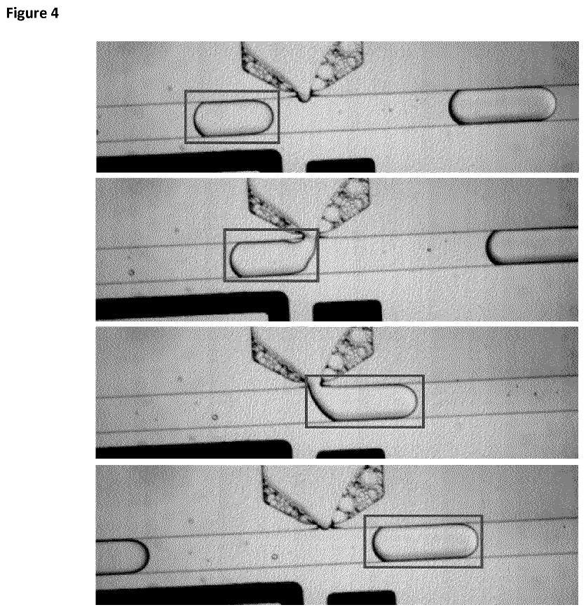

After incubation, the droplets were reinjected into a chip that is capable of nanoinjection. Nanoinjection is employed in order to add the fluorophore directly prior to the start of the assay into droplets. A typical design of nanoinjection device (2) is shown in FIG. 3 and a typical nanoinjection process is pictured in FIG. 4. The flow rate of spacing oil was 0.6 mL/h, the flow rate of droplet reinjection was 0.5 mL/h, and the flow rate of aqueous phase for nanoinjection was 0.1 mL/h. A high voltage with 20,000 V and 20,000 Hz was added to help nanoinjection. Other technologies, such as acoustic wave technology can also be used to add reagents to droplets. The spacing oil phase consists of perfluorocarbon oil. The droplets contain the grown fungi after 48 hours of incubation.

A poly(tetrafluoroethylene) (PTFE) tubing (inner diameter=0.3 mm, outer diameter=0.56 mm) was connected to the outlet (3) of the nanoinjection device (2). The tubing is a delay line (4) in which the droplets that comprise the fungi and the fluorescent molecule are all incubated by moving through a delay line.

### Example 5: Temperature Control of Droplet Incubation

After nanoinjection, the droplets flowed in the PTFE tubing (the delay line (4)). The droplets were continuously moving in the tubing. The length of the tubing in this example was 6 meters, but can also be significantly shorter or longer (e.g. up to 100 m) depending on the incubation time needed. The tubing was incubated at 30° C. in the present example. The temperature setting however can be adapted to the needs of each specific assay. Temperature control was obtained by submerging the tubing containing the droplets for assay incubation into a bed of heated metal beads or in a water bath. Other arrangements like a tubing coil surrounding a peltier element could be another option.

1. Microfluidic method for measuring the viscosity in a micro droplet in a microfluidic system, comprising the steps of
i) providing a micro droplet, wherein the micro droplet comprises a fluid and a fluorescent molecule,
ii) in the microfluidic system, exciting the fluorescent molecule in said micro droplet by applying light to the micro droplet, and
iii) measuring the resulting fluorescence emitted from the micro droplet thereby determining the viscosity of the fluid in the micro droplet.

2. The method according to claim 1, Wherein the fluorescence emitted by the fluorescent molecule depends on the viscosity of the fluid in the micro droplet.

3. Method according to claim 2, wherein
i) the fluorescent molecule undergoes rotational diffusion that is inversely proportional to the viscosity of the fluid in the micro droplet, and/or
ii) the fluorescent molecule is a molecular rotor that forms twisted intra molecular charge transfer upon photo excitation and therefore exhibits two competing de-excitation pathways, the relative intensities of which differ depending on the viscosity.

4. The method according to claim 3, wherein measuring the fluorescence emitted from the micro droplet is performed
i) by determining the fluorescence anisotropy signal when the fluorescent molecule undergoes rotational diffusion, and/or
ii) by measuring the emission intensity when the fluorescent molecule is a molecular rotor.

5. Method according to claim 1, wherein the fluorescent molecule is selected from the group of benzonitrile-based fluorophores benzylidene malononitriles, stilbenes, arimethene dyes, Viscous Blue 1™, Viscous Blue 2™, Viscous Blue 420™, Viscous Green 1™, Viscous Green 2™, Viscous UV™, Viscous Aqua™, Viscous Red™, Viscous VpH™.

6. The method according to claim 1, wherein the fluorescent molecule is introduced into the micro droplet
i) during the formation micro droplet, or
ii) after the formation of the micro droplet by a method such as nanoinjection of picoinjection.

7. The method according to claim 1, wherein the micro droplet has a volume of between 10 pL and 5000 nL.

8. The method according to claim 1, wherein the micro droplet comprises at least one microorganism.

9. Method according to claim 8, wherein the microorganism influences the viscosity of the fluid in the micro droplet.

10. Method according to claim 9, wherein the microorganism influences the viscosity by secreting a substance into the fluid.

11. A method of screening for microorganisms or cells than produce viscosity-modulating compounds comprises the following steps:
a) providing a composition comprising at least one microorganism or cell,
b) optionally subjecting said microorganism Or cell to a reaction that leads to a change in the genetic material of at least one microorganism or cell,
c) encapsulating the microorganism or cell obtained in step a) or b) into a micro droplet, wherein each micro droplet statistically comprises only one microorganism or cell,
d) measuring the viscosity in each of the micro droplets by the method of claim 1 and
e) optionally isolating the micro droplets with the desired viscosity, thereby isolating microorganisms or cells that produce viscosity-modulating compounds with the desired properties.

12. The method of claim 10, wherein step b) is performed by a reaction selected from the group of genetic modification, natural transformation, transduction by phage, conjugation and random mutagenesis, or any other means of modifying the genetic code.

13. The method according to claim 11, wherein the micro droplets with the desired viscosity are isolated by fluorescence activated sorting.

14. The method according to claim 11, wherein the microorganisms are bacteria.

15. (canceled)

16. The method according to claim 5, wherein the benzonitrile-based fluorophore is dimethylamino benonitrile (DMABN).

17. The method according to claim 5, wherein the benzylidene malononitrile is 9-(2,2-dicyanovinyl)julidine (DCVJ).

18. The method according to claim 5, wherein the stilbene is (p-DASPMI).

19. The method according to claim 5, wherein the arimethene dye is crystal violet.
